(19) United States
(12) Patent Application Publication
(10) Pub. No.: US 2022/0228011 A1
(43) Pub. Date: Jul. 21, 2022

(54) METHOD FOR GENERATING A COMPOSITION FOR PAINTS,VARNISHES, PRINTING INKS, GRINDING RESINS, PIGMENT CONCENTRATES OR OTHER COATING SUBSTANCES

(71) Applicant: EVONIK OPERATIONS GMBH, Essen (DE)

(72) Inventors: Oliver KROEHL, Koeln (DE); Gaetano BLANDA, Haltern am See (DE); Stefan SILBER, Krefeld (DE); Sandra BITTORF, Witten (DE); Philipp ISKEN, Altenberge (DE); Inga HUSEN, Dortmund (DE); Ulf SCHOENEBERG, Berlin (DE)

(73) Assignee: EVONIK OPERATIONS GMBH, Essen (DE)

(21) Appl. No.: 17/439,961

(22) PCT Filed: Mar. 13, 2020
(86) PCT No.: PCT/EP2020/056959

§ 371 Date: Sep. 16, 2021

(30) Foreign Application Priority Data
Mar. 18, 2019 (EP) 19163506.9

(51) Int. Cl. C09D 7/80; C09D 7/61; C09D 7/41; C09D 7/45; C09D 7/63; C09D 11/037; G01J 3/46; G06F 16/903

(52) U.S. Cl. CPC C09D 7/80; C09D 7/61; C09D 7/41; C09D 7/45; C08K 3/04; C09D 11/037; G01J 3/463; G06F 16/90335; C09D 7/63

(57) ABSTRACT

The method includes inputting a specification of a desired combination of a first component and a second component of a composition, wherein the components are a solid and a dispersant, via a user interface of a computer system; carrying out a database query with the desired combination of the first component and the second component as the search criterion, wherein in the case where the database query supplies a known composition that meets the search criterion, the known composition is output, wherein in the opposite case, a further database query of a composition including the first component and the second component, serving as the search criterion, is carried out to find a candidate composition that includes one of the two components as well as a substitute component instead of the other component; and carrying out further steps for generating the formulation and outputting the composition.

### TECHNICAL FIELD

The invention relates to a method for generating a composition for paints, varnishes, printing inks, grinding resins, pigment concentrates or other coating substances, the composition comprising solids and dispersants.

### PRIOR ART

Compositions for paint, varnish and printing ink compositions, grinding resins, pigment concentrates and other coating substances are complex mixtures of raw materials. Conventional compositions or recipes or formulations for paints, varnishes, printing inks, grinding resins, pigment concentrates or other coating substances comprise approximately 20 raw materials, also referred to as “components” hereafter. These compositions are, for example, composed of raw materials selected from solids, such as pigments and/or fillers, binders, solvents, resins, hardening agents and various additives, such as thickeners, dispersants, wetting agents, adhesion promoters, defoamers, surface modifying agents, leveling agents, catalytically active additives, such as desiccants and catalysts, and specially acting additives, such as biocides, photoinitiators and corrosion inhibitors.

Thus far, new compositions or formulations having certain, desirable properties are specified based on empirical values and thereafter chemically synthesized and tested. The make-up of a new composition or formulation that meets certain expectations as to the chemical, physical, optical, haptic and other metrologically detectable properties thereof is also almost unpredictable for a person skilled in the art due to the complexity of the interactions. As a result of the wide range of interactions of the raw materials among one another, and associated therewith a plurality of (partially manually executed) experiments and failed tests, this procedure is both time-intensive and cost-intensive.

In general, dispersants (also referred to as dispersing additives) are employed for dispersing solids (for example pigments, fillers or colorants) in liquid media to achieve effective dispersion of the solids for reducing mechanical shearing forces required for dispersion, while achieving the highest possible fill levels. The dispersants support the breakup of agglomerates, and, serving as surface-active materials, wet and/or cover the surfaces of the solids or particles to be dispersed, stabilizing these against undesirable reagglomeration.

Dispersants are also used during the production of paints, varnishes, printing inks, grinding resins, pigment concentrates and other coating substances since the dispersants simplify the incorporation of solids, such as pigments, colorants and fillers which, serving as important composition ingredients, considerably determine the visual appearance and the physicochemical properties of such systems. For optimal utilization, on the one hand, these solids have to be uniformly distributed in the formulations, while, on the other hand, the distribution, once achieved, has to be stabilized.

A plurality of different substances are used today as dispersants for solids. In addition to very simple, low molecular weight compounds, such as lecithin, fatty acids, and the salts and alkylphenol ethoxylates thereof, more complex, high molecular weight structures are also used as dispersants. Here, it is specifically aminofunctional and amidofunctional systems that are widely used.

U.S. Pat. No. 4,224,212, EP-B-0 208 041, WO-A-00/24503 and WO-A-01/21298 describe, for example, dispersants based on polyester-modified polyamines. DE-B-197 32 251 describes polyamine salts and the use thereof as dispersants for pigments and fillers.

### SUMMARY

It is the object of the present invention to provide a method by which the development of a new composition or the development of a reformulation is achieved in a more time-saving and more cost-effective manner.

The object is achieved by the method for generating a composition for paints, varnishes, printing inks, grinding resins, pigment concentrates or other coating substances, wherein the composition comprises solids and dispersants, according to claim 1, and by a corresponding computer system and computer program product. Embodiments of the invention are described in the dependent claims.

Embodiments of the present invention can be freely combined with one another, unless they are mutually exclusive.

In one aspect, the invention relates to a method for generating a composition for paints, varnishes, printing inks, grinding resins, pigment concentrates or other coating substances, the composition being generated by a computer system. The computer system has access to a database in which known compositions and/or formulations are stored.

A “composition” here shall be understood to mean a specification of a chemical product that specifies at least the type of raw materials (“components”) of which the chemical product is formed.

A “formulation” here shall be understood to mean a composition that, in addition to the information of the components, additionally also comprises quantity or concentration information for the particular components.

When mention is made hereafter of compositions, depending on the embodiment, this can also mean that a formulation is present.

For the known compositions, which each comprise solids and dispersants, measuring points for a solids content and a dispersant concentration or a dispersant content are stored in each case in the database.

For example, multiple measuring points can be stored in the database for each known composition. For example, multiple measuring points can be stored for an (established) solids content having a varying dispersant content, or for an (established) dispersant content having a varying solids content. It is part of the method that in each case at least one rheological property and at least one coloristic property are stored in the database for each measuring point of each composition, for example for each tested dispersant concentration having an assigned solids content.

The rheological property can, for example, be the viscosity of the particular composition. The coloristic property can, for example, be the color strength of the particular composition.

The compositions can be paints, varnishes, inkjet ink, pigment pastes, pigment concentrates, grinding resins or other coating substances, such as premixes of certain raw materials (serving as convenience products), dispersions of solids, suspensions, aqueous and solvent-containing compositions, 100% compositions (containing no water and no solvent), or UV-based paint and varnish systems.

Dispersants or surface active compounds within the meaning of the present invention can be chemical amphiphilic, ionic, non-ionic or low molecular weight compounds and/or high molecular weight compounds.

The dispersants or surface active compounds can be polyether-modified fatty acids, polyether-modified fatty acid amidamines, polyether-modified amine derivatives, Jeffamine derivatives, polyether-modified oils, fats and the derivatives thereof, phosphorylated polyether derivatives, in particular those that are fatty alcohol-based, maleic resins or polyether-modified styrene-maleic acid copolymers.

Maleic resins are the Diels-Alder adducts of colophony with maleic or fumaric acid which can still be completely or partly be esterified with polyhydric alcohols. In this way, a very wide range of hard resins having different melting points, functionalities, and resulting solubilities can be produced.

The dispersants or surface active compounds can also be polyesters modified with pigment-affinic groups.

The dispersants or surface active compounds can also be non-ionic ethoxylated sugar-based surfactants, such as polyoxyethylene sorbitan monolaurate, polyoxyethylene sorbitan monopalmitate, polyoxyethylene sorbitan monostearate, polyoxyethylene sorbitan monooleate, and polyoxyethylene sorbitan tristearate.

As an alternative, the dispersants or surface active compounds can be commercially available fatty alcohol ethoxylates, selected from the group of polyalkylene glycol ethers.

It may be provided that the dispersants or surface active compounds have one or more functionalities having an affinity for a pigment surface.

A solid within the meaning of the present invention can, in principle, be any solid organic or inorganic material or carbon black.

Examples of such solids are pigments, fillers, colorants, optical brighteners, ceramic materials, magnetic materials, nanodisperse solids, metals, biocides, agrochemicals, and pharmaceuticals.

A preferred solid is a pigment of any of the recognized classes of pigments which are described, for example, in Third Edition of the Colour Index (1971) and subsequent revised editions and supplements thereto under the chapter entitled “Pigments.”

The pigments are preferably organic or inorganic pigments or carbon black pigments.

For example, iron oxides, chromium oxides or titanium oxides can be mentioned as inorganic pigments.

Suitable organic pigments are, for example, azo pigments, metal complex pigments, anthraquinoid pigments, phthalocyanine pigments, polycyclic pigments, in particular those of the thioindigo, quinacridone, dioxazine, pyrrolopyrrole, naphthalenetetracarboxylic acid, perylene, isoamidolin(on)e, flavanthrone, pyranthrone or isoviolanthrone series.

Gas black, flame black or furnace black can be used as carbon black. These carbon blacks can additionally be post-oxidized and/or pelletized.

Further preferred solids are extenders and fillers, such as talc, kaolin, silicon dioxide, baryte and chalk; particulate ceramic materials, such as alumina, silica, zirconia, titania, silicon nitride, boron nitride, silicon carbide, boron carbide, mixed silicon-aluminum nitrides and metal titanates; particulate magnetic materials, such as the magnetic oxides of transition metals, in particular iron and chromium, such as Gamma-Fe₂O₃, Fe₃O₄ and cobalt-doped iron oxides, calcium oxide, ferrites, in particular barium ferrites and metal particles, in particular metallic iron, nickel, cobalt and alloys thereof, and agrochemicals, such as the fungicides flutriafen, carbendazim, chlorthalonil, and mancozeb.

As mentioned above, the composition for paints, varnishes, printing inks, grinding resins, pigment concentrates or other coating substances comprises solids and dispersants.

A suitable combination of a solid, in particular a pigment, and a dispersant can achieve optimized wetting of the surfaces of the solid with the dispersants, and an optimized distribution and stabilization of the solids, here in particular of the pigments, in the matrix of the composition.

Since the degree of wetting of the pigments is not always known or not directly measurable, the rheological properties, such as the viscosity, and the coloristic properties, such as the color strength, serve as an indicator.

Through the selection of the dispersant and the concentration thereof, the amount used of, for example, expensive solids, such as pigments, can be reduced, without having to accept concessions in the color strength. Furthermore, the viscosity behavior of liquid media that contain solids, such as pigments, is significantly co-determined by the dispersant used. In this regard, especially dispersants are sought which cause as low a viscosity as possible in the liquid paints and varnishes, and also maintain it during storage.

The method according to claim 1 is carried out by initially inputting a specification of a desired combination of a first component and a second component of a composition via a user interface of the computer system. The computer system has access to a database.

A database within the meaning of the present invention can be any memory in which data, in particular structured data, can be stored. The database can be a database that is a text file, a spreadsheet file, a directory in a directory tree, or a database of a database management system (DBMS), such as MySQL or PostgreSQL.

For the method, it is provided that the components, these being the first component and the second component, are a solid and a dispersant.

If the first component is the solid, the second component is the dispersant. Furthermore, if the first component is the dispersant, the second component is the solid.

For better comprehension of the method steps, the first component is referred to hereafter as first component (A), and the second component is referred to as second component (B).

After a specification of a desired combination of the first component (A) and the second component (B) has been input, a database query is carried out with the desired combination of the first component (A) and the second component (B) as the search criterion.

In the case where the database query supplies a known composition that meets the search criterion—first component (A) and second component (B)—the known composition—comprising the first component (A) and the second component (B)—is output. In this way, measuring points for a solids content and a dispersant concentration are already stored for the known composition, wherein the viscosity and the color strength are stored for each measuring point.

In the case where the database query does not supply a known composition that meets the search criterion—first component (A) and second component (B)—another database query of a composition comprising the first component (A) and the second component (B) as the search criterion is carried out to find a candidate composition that comprises one of the two components (first component (A) or second component (B)) as well as a substitute component instead of the other component (second component (B) or first component (A)).

For easier comprehension of the invention, the one of the two components that is present in the candidate composition is referred to as the second component (B) by way of example, and the other component that is not present in the candidate composition is referred to as the first component (A). It shall be noted that the one of the two components could also be referred to as the first component (A), and the other component could also be referred to as the second component (B); however, this is not used in the example for easier comprehension of the invention.

The substitute component is referred to as substitute component (X) in this example for easier comprehension.

In this example, the candidate composition thus comprises the substitute component (X) and the second component (B).

Thereupon a database query is carried out to find a first known comparison composition that comprises the other of the two components, in this example the first component (A), as well as a third component, which is referred to as the third component (C) in this example for easier comprehension.

In this example, the first known comparison composition thus comprises the first component (A) and a third component (C).

In the next step, a database query is carried out to find a second known comparison composition that comprises the substitute component, which was referred to as substitute component (X) in this example for easier comprehension, instead of the other component, which in this example is the first component (A), as well as a third component (C).

In this example, the second known comparison composition thus comprises the substitute component (X) and the third component (C).

In another step, the first and second known comparison compositions are compared by examining a similarity criterion, for example a predefined threshold value, with respect to the measuring points of the first and second known comparison compositions stored in the database.

In the case where the first and second known comparison compositions are assessed as being sufficiently similar, the composition is generated, wherein the substitute component (X) is replaced with the other of the two components, this being the first component (A) in this example, in the candidate composition.

Thereupon, the generated composition is output.

Embodiments of the invention use the surprising finding to advantage that sufficiently similar comparison compositions allow the conclusion that the undesirable substitute component (X) can be replaced with the first component (A) in the candidate composition, and a prediction for a practically usable composition or formulation is thereby obtained. The composition or formulation can subsequently be validated by being produced and examined by a high throughput environment (HTE) apparatus.

For one embodiment of the invention, it is provided that, in the case where multiple candidate compositions are found, the steps of the database queries of the first and second comparison compositions, followed by the comparison of the comparison compositions, are repeated until it is established for at least one of the multiple candidate compositions that the similarity criterion is met, and the composition is generated for this candidate composition, wherein the substitute component (X) is replaced with the other of the two components in this candidate composition. This has the advantage that the selected candidate composition having the highest degree of similarity likely supplies a particularly reliable prediction for a practically usable composition.

For one embodiment, it may be provided that, in the case where multiple candidate compositions are found, the steps of the database queries of the first and second comparison compositions, followed by the comparison of the comparison compositions, are repeated multiple times until it is established for multiple candidate compositions that the similarity criterion is met. In the process, it may be provided as another step that the candidate compositions are sorted using a degree of similarity as the sorting criterion, wherein the candidate composition having the highest degree of similarity is selected, and the step of generating the composition is carried out, wherein the substitute component (X) is replaced with the other of the two components in this candidate composition.

It may be provided for all of the aforementioned embodiments of the invention that a first distance of the extreme values of the rheological property, for example the viscosity, and a second distance of the extreme values of the coloristic property, for example the color strength, in each case with respect to different ratios of the dispersant concentration and solids content, between the first and second known comparison compositions are considered in the degree of similarity.

The distance can, for example, be the length of the shortest connection of the extreme values between the first and second known comparison compositions. The distance can likewise be the length of the rectilinear path between the extreme values in the Euclidean space, a cost function, or a multidimensional distance, wherein the latter is dependent on the number of dimensions (measured parameters, for example viscosity measurements and/or color strength measurements over a varying dispersant concentration at a fixed solids content) or multiple values per measuring point.

It may furthermore be provided for the degree of similarity that measuring points of different storage time periods are stored for the compositions stored in the database, wherein the step of comparing the first and second comparison compositions is carried out for measuring points of different storage time periods so as to ascertain the first and second distances for each of the storage time periods.

So as to test the inhomogeneity or the settling tendency of the solids in the matrix of the composition, the compositions are measured again after one or more storage time periods (for example, after storage of two weeks at 50° C., and after storage of four weeks at 50° C.) to provide information about potential insufficient stabilization, inhomogeneity or settling behavior of the solids in the composition.

Furthermore, it may be provided that the degree of similarity results from a combination of the first and second distances of the extreme values.

The respective two distances between the comparison compositions can thus, for example, be added to one another per dimension (measured parameter, for example viscosity or color strength over, for example, a varying dispersant concentration, at a fixed solids content, or over, for example, a varying solids content, at a fixed dispersant concentration) or be added up overall. If the combination of the distances falls short of or exceeds a predefined threshold value, it may be provided that the degree of similarity is considered to have been met.

As another step, it may be provided that a specification of at least one of the dimensions of the measuring points is input, wherein the selected dimension is overweight in the combination. The selected dimension can, for example, be the viscosity or the color strength.

As an alternative, it may be provided that another step is provided, wherein a specification is input for a value range of the measuring points, wherein, in the step of comparing the first and second known comparison compositions, a similarity criterion is examined with respect to the measuring points of the first and second known comparison compositions stored in the database, and the measuring points are only evaluated in the specified value range. The input can, for example, be a customer request, for example a desired color strength range, a desired storage stability range, or a desired viscosity.

As an alternative, it may be provided that the degree of similarity corresponds to the comparison of the curves of the measuring points, wherein the comparison is carried out by the method of least squares, the squares of the differences, a variance analysis, a string comparison of measuring point ranges or a correlation.

The respective curves of the measuring points of the comparison compositions can be interpreted as two sets of information, the similarity of which can be assessed, for example, by way of a fuzzy retrieval approach, see for example S. Miyamoto (Two approaches for information retrieval through fuzzy associations, IEEE Trans. Syst. Man Cybernet. SMC-19 123-30-1990b Fuzzy Sets in Information Retrieval and Cluster Analysis, 1989, Dordrecht: Kluwer).

It may be provided for all of the aforementioned embodiments of the method that the first known comparison composition, in addition to the other (A) of the two components and the third component (C), comprises at least one further component (for example, D, D+E, D+E+F, . . . ), and the second known comparison component, in addition to the third component (C) and the substitute component (X), comprises one (D), several (D+E; D+E+F) or all (D+E+F, . . . ) of the further components.

It may be provided that the at least one further component is selected from the group consisting of pigments, fillers, colorants, optical brighteners, ceramic materials, magnetic materials, nanodisperse solids, metals, biocides, agrochemicals, pharmaceuticals, binders, solvents, wetting agents, auxiliary leveling agent, hardening agents, and defoamers.

It may furthermore be provided that the solid is an inorganic pigment, an organic pigment, or a carbon black pigment.

The dispersant can be a chemically amphiphilic, ionic, non-ionic or low molecular weight compound and/or high molecular weight compound.

It may furthermore be provided for all of the aforementioned embodiments of the method that, in the case where the one of the two components of the candidate composition is the solid, the solid is an inorganic pigment, an organic pigment, or a carbon black pigment, the third component (C) accordingly likewise is an inorganic pigment, an organic pigment, or a carbon black pigment, and, in the case where the one of the two components of the candidate composition is the dispersant, the dispersant is a chemically amphiphilic, ionic, non-ionic or low molecular weight compound and/or high molecular weight compound, the third component (C) accordingly likewise is a chemically amphiphilic, ionic, non-ionic or low molecular weight compound and/or high molecular weight compound.

In this way, the one of the two components and the third component are similar, for example in terms of the properties thereof, such as color, size, structure, weight, charge and the like.

It may be provided that the measuring points of the first and second known comparison compositions or comparison formulations stored in the database were obtained by a preceding examination of the rheological property, for example the viscosity, and of the coloristic property, for example the color strength, in relation to a dispersant concentration and a solids content using an apparatus for carrying out a production process of a formulation for paints, varnishes, printing inks, grinding resins, pigment concentrates or other coating substances, and an examination of a formulation for paints, varnishes, printing inks, grinding resins, pigment concentrates or other coating substances.

A suitable apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances is, for example, the high throughput environment (HTE) apparatus made by Chemspeed Technologies AG, which is described in WO 2017/072351 A2, or the apparatus for high throughput research (high throughput apparatus) described in EP 2 420 817 B1 in paragraph [0054].

It may furthermore be provided that the computer system communicates via a communication interface with the database and/or an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, wherein the communication interface is implemented by USB, Ethernet, WLAN, LAN, Bluetooth or another network interface.

The generated composition or formulation can be output on the user interface of the computer system. In addition or as an alternative, it may be provided that a formulation is stored for the generated composition or that, proceeding from the generated composition, one or more formulations are generated, for example by a test planning program, wherein the stored formulation or the formulation generated by the test planning program is output to a processor, wherein the processor controls an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances.

The apparatus comprises at least two processing stations, wherein the at least two processing stations are connected to one another via a transport system on which self-propelled transport vehicles (for example a vessel suitable for receiving the formulation or a substrate, so-called panel, to which the formulation can be applied) can travel between the processing stations for transporting the components of the composition and/or of the produced formulation. The produced formulation can, for example, be transported between the processing stations in a vessel suitable for receiving the formulation, in particular a vessel made of plastic or glass. Likewise, the produced formulation can be applied onto a substrate or onto a panel, wherein the substrate or panel, together with the applied formulation, is transported between the processing stations. The substrate or panel can be made of any material to which the formulation can be applied by a user, in particular glass, plastic, concrete, wood, metal, stone, and the like. The processor activates the apparatus to produce the generated composition or formulation, wherein the production of the generated formulation and an examination of the rheological property and of the coloristic property take place in the at least two processing stations. For example, the examination of the rheological property and of the coloristic property can take place. Thereupon, the results of the examination of the rheological property and of the coloristic property are output on the user interface of the computer system and/or the results of the examination of the rheological property and of the coloristic property are stored in the database. The results or measuring points of the formulation stored in the database can thus be used for subsequent processes.

A further aspect of the invention is a computer system for generating a composition for paints, varnishes, printing inks or other coating substances, comprising a database and a user interface, wherein the computer system is configured to carry out the above-described method.

Another aspect of the invention is a computer program, a digital storage medium or a computer program product including instructions executable by a processor to carry out the above-described method.

Another aspect of the invention is a system comprising an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, wherein the apparatus comprises at least two processing stations, wherein the at least two processing stations are connected to one another via a transport system on which self-propelled transport vehicles can travel between the processing stations for transporting the components of the composition and/or the produced formulation, wherein both the production of the formulation and an examination of a rheological property, for example viscosity, and of a coloristic property, for example color strength, in relation to a dispersant concentration and a solids content of each composition take place in the at least two processing stations, and a computer system for generating a composition for paints, varnishes, printing inks, grinding resins, pigment concentrates or other coating substances, comprising a database and a user interface, wherein the computer system is configured to carry out the above-described method.

### BRIEF DESCRIPTION OF THE DRAWINGS

Embodiments of the invention are described in greater detail in an exemplary manner in the following figures:

FIG. 1 shows a flow chart of a method for generating a composition for paints, varnishes, printing inks, grinding resins, pigment concentrates or other coating substances, the composition for paints, varnishes, printing inks, grinding resins, pigment concentrates or other coating substances comprising solids and dispersants;

FIG. 2 shows a block diagram of a system comprising a computer system, a database, and a HTE installation; and

FIGS. 3A-3C show three diagrams for compositions, each comprising ferric oxide red pigments and dispersants, measuring values for the viscosity and the color strength being provided for compositions that comprise the same dispersant.

### DETAILED DESCRIPTION

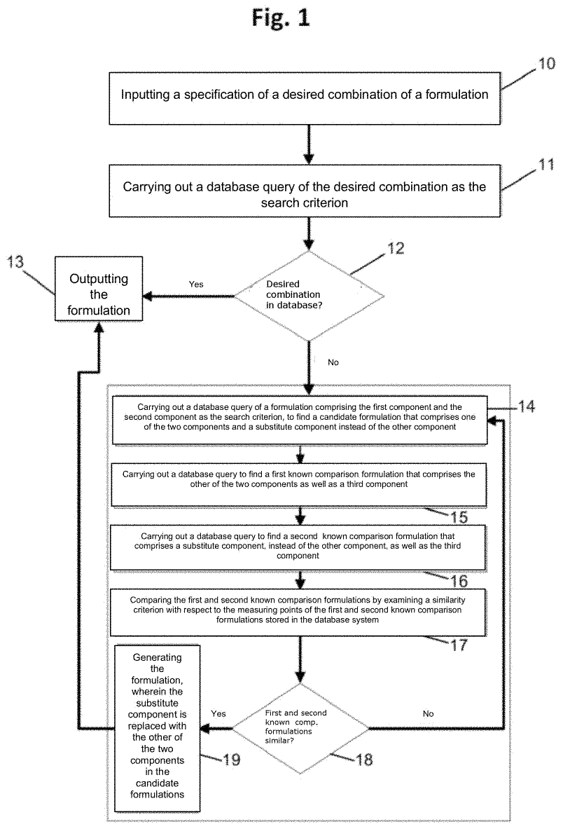

FIG. 1 shows a flow chart of a method for generating a composition for paints, varnishes, printing inks, grinding resins, pigment concentrates or other coating substances.

The method can be executed by a computer system, for example a control computer, a notebook, a standard computer, a tablet computer, or a smart phone.

The method can typically be used in the context of a chemical laboratory. A series of individual analytical devices and a high throughput environment (HTE) apparatus, this being an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, are present in the laboratory. The HTE apparatus includes a plurality of units and modules, which are able to analyze and measure different chemical or physical parameters of substances and substance mixtures and which can combine and synthesize a plurality of different chemical products, based on a recipe or formulation input by a user.

The computer system has access to a database, in which known compositions or formulations are stored and in which respective measuring points of the particular formulation for a solids content and a dispersant concentration are stored for combinations of a solid and a dispersant of known formulations. A rheological property and a coloristic property are stored for each measuring point of the particular formulation.

The rheological property can be the viscosity, and the coloristic property can be the color strength. Both the rheological property and the coloristic property can have been ascertained by way of the HTE installation.

In a first step 10, a user, for example a researcher, who wants to develop new compositions or formulations inputs a specification of a desired combination of a first component and of a second component of a composition via a user interface 101 of the computer system 100.

It is provided for the method according to the invention that the first component and the second component are a solid and a dispersant.

In a next step 11, a database query is carried out with the desired combination of the first component and the second component as the search criterion.

If the database query supplies a known composition or formulation that meets the search criterion, the known composition or formulation is output in a further step 13.

If the database query does not supply a known composition or formulation that meets the search criterion, another database query of a composition or formulation comprising the first component and the second component as the search criterion is carried out in a further step 14, to find a candidate composition or candidate formulation that comprises one of the two components as well as a substitute component instead of the other component.

For this purpose, a database query is carried out in a further step 15 to find a first known comparison composition or comparison formulation that comprises the other of the two components as well as a third component.

Moreover, a database query is carried in a further step 16 to find a second known comparison composition or comparison formulation that comprises a substitute component, instead of the other component, as well as the third component.

After steps 15 and 16, a comparison of the first and second known comparison compositions or comparison formulations is carried out in a further step 17 by examining a similarity criterion, for example a predefined threshold value, with respect to the measuring points of the first and second known comparison compositions or comparison formulations stored in the database, wherein, in the case where the first and second known comparison compositions are assessed as being sufficiently similar, the composition or formulation is generated in a subsequent step 19, wherein the substitute component is replaced with the other of the two components in the candidate composition or candidate formulation, and the generated composition or formulation is output, see step 13.

In the case where the first and second known comparison compositions or comparison formulations are assessed as not being sufficiently similar, steps 14 to 18 are carried out again.

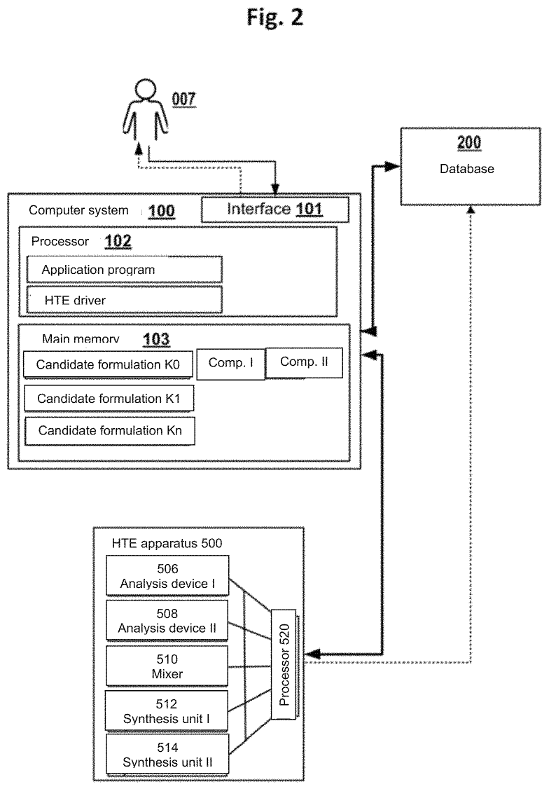

FIG. 2 shows a block diagram of a system comprising a computer system 100, a database 200, and an apparatus for carrying out a production process of a composition for paints, varnishes, printing inks, grinding resins, pigment concentrates or other coating substances and an examination of a composition for paints, varnishes, printing inks, grinding resins, pigment concentrates or other coating substances (HTE apparatus) 500.

The essential functions of the components of the system and the components thereof were already described with respect to FIG. 1. The computer system 100 can be a notebook, a standard computer, a tablet computer, or a smart phone, for example, which is operated by a user 007. The user 007 enters, for example, the input of a specification of a desired combination of a composition or formulation via the interface 101 of the computer system (see also step 10 in FIG. 1). The compositions described hereafter can also be formulations.

The computer system 100 has access to a database, in which known compositions are stored and in which respective measuring points of the particular composition for a solids content and a dispersant concentration are stored for combinations of a solid and a dispersant of known compositions.

In addition, the computer system 100 comprises a processor 102 including an application program as well as a driver for controlling a processor 520 of the HTE apparatus 500. As is shown, the HTE apparatus 500 comprises a plurality of units and modules 506 to 514, which are able to analyze and measure different chemical or physical parameters of substances and substance mixtures. For example, the unit 506 is an analytical device that is able to adjust and determine the viscosity. For example, the unit 508 is an analytical device that is able to ascertain color values based on an integrated spectrophotometer (using a so-called L*a*b value).

In addition, the computer system 100 comprises a main memory 103 in which the candidate composition K0 and further candidate compositions K1 to Kn, where n≥2 can apply, and the comparison compositions Comp I, Comp II can be stored.

As is shown, the computer system 100 can both store data, for example compositions and/or measuring values and properties of components, on the database 200 and retrieve data from the database 200. The computer system 100 can likewise transmit data, for example requests, to the HTE apparatus 500 and receive the results. The results can thereupon be forwarded to the database system 200 and/or (via the interface 101) to the user 007. In addition, it is provided that the results that were ascertained by way of the HTE apparatus 500 can be directly entered into the database 200.

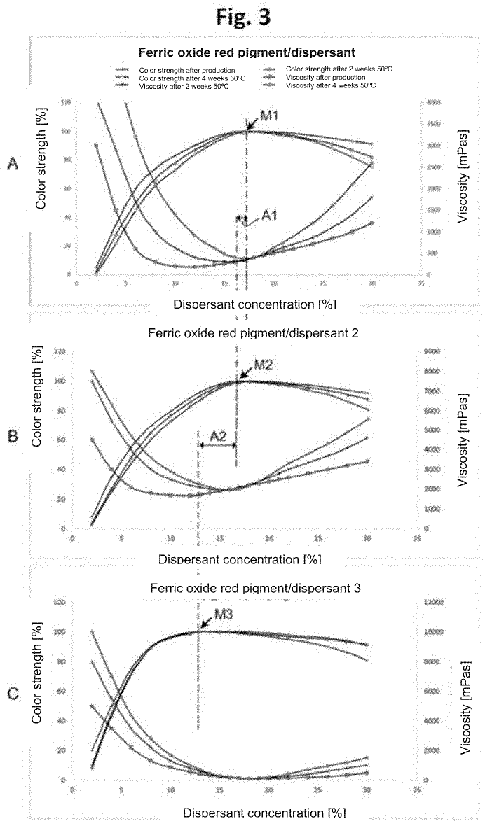

FIG. 3 shows three diagrams (FIG. 3A, FIG. 3B and FIG. 3C) for compositions that each comprise ferric oxide red pigments and dispersants.

FIG. 3A shows measuring values for the color strength and the viscosity of compositions that comprise a dispersant 1 and ferric oxide red pigments in relation to different or varying dispersant concentrations, at a fixed content of ferric oxide red pigments, in the form of curves.

FIG. 3B shows measuring values for the color strength and the viscosity of compositions that comprise a dispersant 2 and ferric oxide red pigments in relation to different or varying dispersant concentrations, at a fixed content of ferric oxide red pigments, in the form of curves.

FIG. 3C shows measuring values for the color strength and the viscosity of compositions that comprise a dispersant 3 and ferric oxide red pigments in relation to different or varying dispersant concentrations, at a fixed content of ferric oxide red pigments, in the form of curves.

The dispersants 1, 2 and 3 shown in FIGS. 3A to 3C are different aqueous solutions of a copolymer comprising pigment-affinic groups. The dispersants 1, 2 and 3 differ from one another either in the copolymer, in the appearance thereof (clear, cloudy or color), the acid number [mg KOH/g], the viscosity [mPa·s at 25° C.], the flash point [° C.] and/or the melting point [° C.]. In FIGS. 3A to 3C, measuring values of the viscosity and of the color strength are shown for compositions having varying dispersant contents, wherein the dispersant content varies between 1% and 30% in relation to a fixed content of ferric oxide red pigments (65% here). The remainder of the composition consists of the further composition, here additives, defoamers, water, and biocides.

FIGS. 3A, 3B and 3C additionally show the measuring values for the color strength and the viscosity of each of the compositions (comprising the same dispersant, but in varying contents) after different storage time periods. The different storage time periods are: i) after production (see measuring points denoted by the cross symbol for the color strength, and measuring points denoted by the square symbol for the viscosity); ii) after storage of the composition for two weeks at 50° C. (see measuring points denoted by the triangle symbol for the color strength, and measuring points denoted by the star symbol for the viscosity); and iii) after storage of the composition for four weeks at 50° C. (see measurement points denoted by the diamond symbol for the color strength, and measurement points denoted by the circle symbol for the viscosity).

The measuring values for the color strength and the viscosity were measured by an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances (HTE apparatus made by Chemspeed Technologies AG, described in WO 2017/072351 A2) and stored in the database. The storage stability measurements were measured based on the inhomogeneity or settling tendency, wherein a plate/spindle measures the normal force during a downward movement, thereby detecting inhomogeneities, such as syneresis or sediment. The viscosity measurements of the compositions, including viscosity adjustment by dilution, were likewise carried out by the HTE apparatus by determining the viscosity of the compositions in a transport vehicle (vessel made of glass) by way of a blade agitator. The color values of the compositions were likewise measured by the HTE apparatus, based on an integrated spectrophotometer (so-called L*a*b value).

For the method according to the invention, it may be provided, for example, that the first known comparison composition (see FIG. 1, step 15) is one or more of the compositions from FIG. 3A. The ferric oxide red pigment would thus be the third component, and the dispersant 1 would be the other of the two components. It may furthermore be provided that the second known comparison composition (see FIG. 1, step 16) is one or more of the compositions from FIG. 3B. The ferric oxide red pigment would thus be the third component, and the dispersant 2 would be the substitute component.

According to the method according to the invention, a comparison is then carried out between the first and second known comparison compositions by examining a similarity criterion, for example a previously established threshold value, with respect to the measuring points of the first and second known comparison compositions stored in the database.

For example, a first distance of the extreme values of the viscosity and/or a second distance of the extreme values of the color strength, in each case in relation to a dispersant concentration and a solids content, between the first (FIG. 3A) and the second (FIG. 3B) known comparison compositions can be considered in the degree of similarity.

As is apparent from FIG. 3A compared to FIG. 3B, the color strength for all measured compositions is at the maximum thereof (indicated by M1 in FIG. 3A, and indicated by M3 in FIG. 3B), here at approximately 100%, for the compositions having a dispersant content of 16% to 19%. Since the maxima M1, M2 of the color strength between FIGS. 3A and 3B for the dispersant content are not spaced apart from one another, or are not spaced far apart from one another (see distance A1), it can be provided that the first (FIG. 3A) and the second (FIG. 3B) known comparison compositions are assessed as being sufficiently similar. In this way, the distance of the maxima can be, for example, at approximately 2% of the dispersant concentration here. For example, the comparison compositions are assessed as being similar when the similarity criterion, serving as a threshold value, specifies that the distance should be less than 3% of the dispersant concentration. As is shown, it is likewise apparent from FIGS. 3A and 3B that the intersecting points of the storage time period curves, by comparison, are likewise not spaced far apart (<3% of the dispersant concentration) from one another for the compositions of FIGS. 3A and 3B. In this way, these intersecting points can likewise be considered as the distance in the degree of similarity, whereby it may be provided that the first (FIG. 3A) and the second (FIG. 3B) known comparison compositions are assessed as being sufficiently similar. The degree of similarity can likewise correspond to the comparison of the curves of the measuring points, wherein the comparison is carried out by the method of least squares, the squares of the differences, a variance analysis, a string comparison of measuring point ranges or a correlation. As is shown here for the compositions comprising different dispersants in FIGS. 3A and 3B, the curves of the measuring values of the compositions after production behave very similarly, and can thus be assessed as being sufficiently similar.

Since the first and second known comparison compositions were assessed as being sufficiently similar, according to the claimed method the composition can be generated, wherein the substitute component (dispersant 2) is replaced with the other of the two components (dispersant 1) in the candidate composition, whereupon the generated composition is output.

For the method according to the invention, it may furthermore be provided, for example, that the first known comparison composition (see FIG. 1, step 15) is one or more of the compositions from FIG. 3A. The ferric oxide red pigment would thus be the third component, and the dispersant 1 would be the other of the two components, and the second known comparison composition (see FIG. 1, step 165) would be one or more of the compositions from FIG. 3C. The ferric oxide red pigment would thus be the third component, and the dispersant 3 would be the substitute component.

As is apparent from FIG. 3C, the color strength is at the maximum M3 thereof, here at approximately 100%, for the compositions having a dispersant content of approximately 13%, wherein the viscosity [mPas} is at the minimum thereof at a dispersant content of approximately 15 to 23%. Since at least the maxima M1 and M2 of the color strength from FIGS. 3A and 3B are spaced (rather) far apart from the maximum of the color strength M3 for FIG. 3C (the distance is approximately 5% of the dispersant concentration), it may be provided that the first (FIG. 3A) and the second (FIG. 3C) known comparison compositions are assessed as not being sufficiently similar. It is likewise apparent from FIGS. 3A and 3C that the intersecting points of the storage time period curves, at least for the color strength, by comparison are likewise spaced (rather) far apart from one another. In this way, these intersecting points can likewise be considered as the distance in the degree of similarity, whereby it may be provided in this case that the first (FIG. 3A) and the second (FIG. 3C) known comparison compositions are assessed as not being sufficiently similar.

### LIST OF REFERENCE NUMERALS

10-19 steps
007 user
100 computer system
101 interface
102 processor
103 main memory
200 database
500 apparatus for carrying out a production process and an examination of a formulation for paints, varnishes, printing inks, grinding resins, pigment concentrates or other coating substances (HTE apparatus)
506 analytical device
508 analytical device
510 mixer
512 synthesis unit
514 synthesis unit
520 processor

M1 maximum of the color strength for the composition in FIG. 3A
M2 maximum of the color strength for the composition in FIG. 3B
M3 maximum of the color strength for the composition in FIG. 3C
A1 distance between M1 and M2 when comparing FIGS. 3A and 3B
A2 distance between M2 and M3 when comparing FIGS. 3B and 3C

1. A method for generating a composition for paints, varnishes, printing inks, grinding resins, pigment concentrates or other coating substances, the composition for paints, varnishes, printing inks, grinding resins, pigment concentrates or other coating substances comprising solids and dispersants, the composition being generated by a computer system, the computer system having access to a database, in which known compositions are stored and in which respective measuring points of the particular composition for a solids content and a dispersant concentration are stored for combinations of a solid and a dispersant of known compositions, a rheological property and a coloristic property being stored for each measuring point of the particular composition,
comprising the following steps:
a. inputting a specification of a desired combination of a first component and a second component of a composition, the components being a solid and a dispersant, via a user interface of the computer system;
b. carrying out a database query with the desired combination of the first component and the second component as the search criterion,
(1) in the case where the database query supplies a known composition that meets the search criterion, the known composition being output;
(2) in the opposite case, a further database query of a composition comprising the first component and the second component, serving as the search criterion, being carried out to find a candidate composition that comprises one of the two components as well as a substitute component instead of the other component,
i. a database query being carried to find a first known comparison composition that comprises the other of the two components as well as a third component;
ii. a database query being carried to find a second known comparison composition that comprises a substitute component, instead of the other component, as well as the third component;
iii. comparing the first and second known comparison compositions by examining a similarity criterion with respect to the measuring points of the first and second known comparison compositions stored in the database, in the case where the first and second known comparison compositions are assessed as being sufficiently similar
iv. the composition being generated, the substitute component being replaced with the other of the two components in the candidate composition, and
v. the generated composition being output.

2. The method according to claim 1, wherein, in step b., a plurality of candidate compositions are found, and steps i. to iii. are repeated until it is established for at least one of the plurality of candidate compositions that the similarity criterion is met, and step iv. is carried out for this candidate composition.

3. The method according to claim 1, wherein, in step b., a plurality of candidate compositions are found, and steps i. to iii. are repeated multiple times until it is established for a plurality of candidate compositions that the similarity criterion is met, comprising the further step of sorting the candidate compositions, the candidate compositions being sorted using a degree of similarity as the sorting criterion, the candidate composition that have the highest degree of similarity being selected, and step iv. being carried out for this candidate composition.

4. A method according to claim 1, wherein a first distance of the extreme values of the rheological property and a second distance of the extreme values of the coloristic property, in each case in relation to a dispersant concentration and a solids content, between the first and second known comparison compositions are considered in the degree of similarity.

5. The method according to claim 4, wherein measuring points of different storage time periods are stored for the formulations stored in the database, step being carried out for measuring points of different storage time periods to ascertain the first and second distances for each of the storage time periods.

6. The method according to claim 4, wherein the degree of similarity results from a combination of the first and second distances of the extreme values.

7. The method according to claim 5, comprising the further step of inputting a specification of at least one of the dimensions of the measuring points, the selected dimension being overweight in the combination.

8. A method according to claim 1, comprising the further step of inputting the specification for a value range of the measuring points, in step b. iii. the measuring points only being evaluated in the specified value range.

9. A method according to claim 1, wherein the degree of similarity corresponds to the comparison of the curves of the measuring points, wherein the comparison is carried out by the method of least squares, the squares of the differences, a variance analysis, a string comparison of measuring point ranges or a correlation.

10. A method according to claim 1, wherein
in step b. i., the first known comparison composition comprises further components, in addition to the other of the two components and the third component;
in step b. .ii, the second known comparison composition comprises one, more or all of the further components, in addition to the third component and the substitute component.

11. A method according to claim 1, wherein the further components are selected from the group consisting of pigments, fillers, colorants, optical brighteners, ceramic materials, magnetic materials, nanodisperse solids, metals, biocides, agrochemicals, pharmaceuticals, binders, solvents, wetting agents, auxiliary leveling agent, hardening agents, and defoamers.

12. A method according to claim 1, wherein the solid is an inorganic pigment, an organic pigment, or a carbon black pigment.

13. A method according to claim 1, wherein the dispersant is a chemically amphiphilic, ionic, non-ionic or low molecular weight compound and/or high molecular weight compound.

14. A method according to claim 1, wherein
in the case where the one of the two components of the candidate composition is the solid, the solid is an inorganic pigment, an organic pigment or a carbon black pigment, the third component accordingly likewise is an inorganic pigment, an organic pigment or a carbon black pigment; and
in the case where the one of the two components of the candidate composition is the dispersant, the dispersant is a simple, low molecular weight compound and/or a complex, high molecular weight compound, the third component accordingly likewise is a simple, low molecular weight compound or a complex, high molecular weight compound.

15. A method according to claim 1, wherein the measuring points of the first and second known comparison compositions stored in the database were obtained by a preceding examination of the rheological property and the coloristic property in relation to a dispersant concentration and a solids content using an apparatus for carrying out a production process of a formulation for paints, varnishes, printing inks, grinding resins, pigment concentrates or other coating substances, and an examination of a formulation for paints, varnishes, printing inks, grinding resins, pigment concentrates or other coating substances.

16. A method according to claim 1, wherein the computer system communicates via a communication interface with the database and/or an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, the communication interface being implemented by USB, Ethernet, WLAN, LAN, Bluetooth or another network interface.

17. A method according to claim 1, wherein, after step b. or after step b. v, the generated composition is output to the user interface of the computer system.

18. A method according to claim 1, wherein, after step b. or after step b. v, the generated composition is output to a processor, the processor controlling an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, the apparatus comprising at least two processing stations, the at least two processing stations being connected to one another via a transport system on which self-propelled transport vehicles can travel between the processing stations for transporting the components of the formulation and/or the produced formulation, the processor activating the apparatus for producing the generated composition, the production of the generated composition and an examination of the rheological property and of the coloristic property, in relation to the dispersant concentration and the solids content of the formulation, taking place in the at least two processing stations, whereupon the results of the examination of the rheological property and of the coloristic property in relation to the dispersant concentration and the solids content of the formulation are output to the user interface and/or the results of the examination of the rheological property and of the coloristic property in relation to the dispersant concentration and the solids content of the formulation are stored in the database.

19. A computer system for generating a composition for paints, varnishes, printing inks, grinding resins, pigment concentrates or other coating substances, comprising a database and a user interface, the computer system being configured to carry out a method according to claim 1.

20. A non-transitory digital storage medium, including instructions executable by a processor to carry out a method according to claim 1.

21. A system, comprising:
an apparatus for carrying out a production process of a formulation for paints, varnishes, printing inks, grinding resins, pigment concentrates or other coating substances and an examination of a formulation for paints, varnishes, printing inks, grinding resins, pigment concentrates or other coating substances, the apparatus comprising at least two processing stations, the at least two processing stations being connected to one another via a transport system on which self-propelled transport vehicles can travel between the processing stations for transporting the components of the formulation and/or the produced formulation, both the production of the formulation and an examination of the rheological property and of a coloristic property in relation to a dispersant concentration and a solids content of each formulation taking place in the at least two processing stations; and
a computer system according to claim 19.
